(19) United States
(12) Patent Application Publication
(10) Pub. No.: US 2012/0035347 A1
(43) Pub. Date: Feb. 9, 2012

(54) INTERFERON-ALFA SENSITIVITY BIOMARKERS

(75) Inventor: Antoine Jean Yver, Belle Mead, NJ (US)

(73) Assignee: Schering Corporation, Kenilworth, NJ (US)

(21) Appl. No.: 13/264,513

(22) PCT Filed: Apr. 13, 2010
(86) PCT No.: PCT/US10/30867

§ 371 Date: Oct. 14, 2011

Related U.S. Application Data
(60) Provisional application No. 61/169,235, filed on Apr. 14, 2009.

(51) Int. Cl. C12Q 1/06; C07K 14/56

(52) U.S. Cl. 530/351; 435/39

(57) ABSTRACT

The present invention provides biomarkers of sensitivity to interferon alfa (IFN-α). These IFN-α sensitivity biomarkers are useful, inter alia, to identify patients who are most likely to benefit from treatment with pharmaceutical compositions of IFN-α, in methods of treating patients having a disease susceptible to treatment with interferon alfa, and in methods for selecting the most appropriate therapy for such patients.

### FIELD OF THE INVENTION

The present invention relates to biomarkers that are predictive of a beneficial response to therapy with an interferon alfa.

### BACKGROUND OF THE INVENTION

Identification of any publication in this section or any section of this application is not an admission that such publication is prior art to the present invention.

The type I interferon alfa (IFN-α) family of proteins exhibit clinically important antiviral, antiproliferative and immunomodulatory activities, and various IFN-α proteins have been approved for treating a variety of diseases, including hepatitis and cancers. Due to the short plasma half-life of the originally approved IFN-α proteins, longer-acting versions have been developed: in particular, peginterferon alfa-2a, marketed by Hoffman-La Roche (Nutley, N.J.) under the trade name PEGASYS®; peginterferon alfa-2b, marketed by Schering-Plough (Kenilworth, N.J.) under the trade name Peglntron®; and Albuferon®, a fusion between human serum albumin and interferon alfa-2b, which is in late-stage clinical development by Human Genome Sciences.

IFN-α proteins affect a variety of cellular functions, including DNA replication and RNA and protein synthesis, in both normal and abnormal cells. Thus, cytotoxic effects of IFN-α therapy are not restricted to tumor or virus infected cells but are also manifested in normal, healthy cells as well. As a result, undesirable, but typically reversible, side effects arise during IFN-α therapy, particularly when high doses are required to achieve a therapeutic effect. For example, administration of IFN-α proteins can lead to reduced red blood cell, white blood cell and platelet counts, and high doses commonly produce flu-like symptoms (e.g., fever, fatigue, headaches and chills), gastrointestinal disorders (e.g., anorexia, nausea and diarrhea), mood changes and alteration of liver enzymes.

Such side effects can be particularly of concern due to the long treatment times typically required with IFN-α-based therapy. For example, the recommended duration of peginterferon alfa/ribavirin combination therapy for hepatitis C virus (HCV) infection is between 24 and 48 weeks, depending on HCV genotype and baseline viral load. The treatment duration for certain cancer indications may be even longer, as evidenced by a recently completed clinical trial of peginterferon alfa-2b as adjuvant therapy for resected stage III melanoma, in which the patients were treated with 6 μg/kg peginterferon alfa-2b a week subcutaneously for 8 weeks (induction phase), followed by 3 μg/kg per week subcutaneously for an intended treatment duration of 5 years (maintenance phase) (Eggermont A. M. M. et al., Lancet 372:117-126 [2008]).

In addition to the potential for problematic side effects, the therapeutic effect of IFN-α therapy cay vary widely among patients with a particular disease. For example, combination peginterferon alfa-2b/ribavirin therapy produces a sustained viral response (SVR) rate of between approximately 20% and 93% in various patient groups defined by HCV genotype and baseline viral load. Similarly, Eggermont et al., supra, reported better clinical outcomes for patients with earlier stage III melanoma than for patients with later stage disease, in particular an overall risk reduction of relapse of approximately 18-25%.

Thus, in view of the side effect and variable response and sensitivity profiles observed with IFN-α therapy, a need exists for a way of identifying patients who are most likely to benefit from IFN-α therapy. The present invention addresses this need.

### SUMMARY OF THE INVENTION

The present invention provides biomarkers of sensitivity to IFN-α treatment. These IFN-α sensitivity biomarkers, which are biomarkers of an individual's pre-treatment immune status, fall within two classes: biomarkers of a heightened pre-treatment, non-specific inflammatory state, such as elevated baseline levels of C-reactive protein or other acute phase proteins, and biomarkers of an on-treatment adverse reaction, such as reduced on-treatment levels of neutrophils or certain other blood cell types. The biomarkers of the present invention may be used to identify individuals who are most likely to benefit from IFN-α therapy for any disease susceptible to treatment with an IFN-α.

Thus, in one embodiment, the invention provides a composition comprising an interferon alfa (IFN-α) for treating an individual having a disease susceptible to treatment with the IFN-α and a positive test for at least one IFN-α sensitivity biomarker.

In another embodiment, the invention provides the use of an IFN-α in the manufacture of a medicament for treating an individual having a disease susceptible to treatment with the IFN-α and a positive test for at least one IFN-α sensitivity biomarker.

In yet a further embodiment, the invention provides a method of predicting an individual's response to therapy with an IFN-α. The method comprises obtaining a blood sample from the individual, assaying the blood sample for the presence of at least one interferon sensitivity biomarker, and making a prediction based on the results of the assaying step. If the results are positive for the presence of the assayed biomarker, the prediction is that the individual is likely to achieve a beneficial response, and if the results are negative for the presence of the assayed biomarker, the prediction is that the individual is not likely to achieve a beneficial response.

The invention also provides a screening method for selecting individuals for initial treatment or continued treatment with an IFN-α from a group of individuals having a disease susceptible to treatment with the IFN-α. This screening method comprises testing each member of the disease group for the presence of at least one IFN-α sensitivity biomarker and selecting for treatment at least one individual testing positive for the interferon sensitivity biomarker.

In a still further embodiment, the invention provides method of selecting a therapy for treating an individual having a disease susceptible to treatment with the IFN-α, comprising testing the individual for the presence of at least one IFN-α sensitivity biomarker and selecting a therapy based on the results of the testing step, wherein if the individual tests positive for the IFN-α sensitivity biomarker, the selected therapy comprises initial treatment or continued treatment with the IFN-α and if the individual tests negative for the interferon sensitivity biomarker, the selected therapy comprises the IFN-α in combination with at least one other therapeutic agent that is not an IFN-α or the selected therapy excludes IFN-α-based therapy.

In each of the above embodiments, the IFN-α sensitivity biomarker is an elevated pre-treatment level of an acute phase protein, a reduced on-treatment level of high sensitivity CRP (hsCRP) or a reduced on-treatment level of at least one blood cell type selected from the group consisting of: neutrophils, erythrocytes, platelets, monocytes, eosinophils, and basophils. Preferred IFN-α sensitivity biomarkers for use in guiding the treatment of high-risk melanoma patients are an elevated pre-treatment hsCRP level and neutropenia classified as grade 2 or greater. In some preferred embodiments, the IFN-α is a pegylated IFN-α-2a or IFN-α-2b, and in particularly preferred embodiments, the IFN-α is PegIntron® (peginterferon alfa-2b).

### BRIEF DESCRIPTION OF THE DRAWINGS

FIG. 1 is a Kaplan-Meier plot (KM) representation of the relapse-free survival (RFS) of high-risk melanoma patients from the pivotal study EORTC 18991 described in Eggermont et al., supra who were treated with PegIntron® (peginterferon alfa-2b) and either experienced Grade 2 or worse neutropenia (solid line, denoted as W2+ NEU) or did not experience neutropenia or whose neutropenia never reached grade 2 or worse (dotted line, denoted W/o2+ NEU). On the x axis is the time from randomization, in months and on the y axis is the actuarial probability of staying alive and free from relapse. Further details are found in the Examples.

FIG. 2 is a Kaplan-Meier plot (KM) representation of the overall survival (OS) of high-risk melanoma patients from the pivotal study EORTC 18991 described in Eggermont et al., supra who were treated with PegIntron® (peginterferon alfa-2b) and either experienced Grade 2 or worse neutropenia (solid line, denoted as W2+ NEU) or did not experience neutropenia or whose neutropenia never reached grade 2 or worse (dotted line, denoted W/o2+ NEU). On the x axis is the time from randomization, in months and on the y axis is the actuarial probability of staying alive. Further details are in the Examples.

FIG. 3 is a Kaplan-Meier plot (KM) estimate of the time from randomization to first observation of a neutropenia of grade 2 or above in severity experienced by high-risk melanoma patients from the pivotal study EORTC 18991 described in Eggermont et al., supra who were treated with PegIntron® (peginterferon alfa-2b). On the x axis is the time from randomization, in months, and on the y axis is the KM estimate of the actuarial proportion of not having observed the event first grade 2 or above neutropenia. Further details are in the Examples.

### DETAILED DESCRIPTION OF THE INVENTION

### I. Definitions.

So that the invention may be more readily understood, certain technical and scientific terms are specifically defined below. Unless specifically defined elsewhere in this document, all other technical and scientific terms used herein have the meaning that would be commonly understood by one of ordinary skill in the art to which this invention belongs when used in similar contexts as used herein.

As used herein, including the appended claims, the singular forms of words such as “a,” “an,” and “the,” include their corresponding plural references unless the context clearly dictates otherwise.

“About” when used to modify a numerically defined parameter, e.g., the dosage for a therapeutic agent discussed herein, means that the parameter may vary by as much as 10% above or below the stated numerical value for that parameter. For example, a dosage of about 3 μg/kg of PEG12K-interferon alfa-2b, used in the treatment of melanoma patients, could vary between 2.7 and 3.3 μg/kg.

“Beneficial result” means a desired clinical result of treatment with an IFN-α, including but not limited to: alleviation of one or more disease symptoms, diminishment of extent of disease, stabilized (i.e., not worsening) state of disease, slowing of disease progression, amelioration or palliation of a disease state, prolonging survival (as compared to expected survival if not treated), relapse-free survival, remission (whether partial or total) and cure (i.e., elimination of the disease).

“Consists essentially of” and variations such as “consist essentially of” or “consisting essentially of” as used throughout the specification and claims, indicate the inclusion of any recited elements or group of elements, and the optional inclusion of other elements, of similar or different nature than the recited elements, which do not materially change the basic or novel properties of the specified dosage regimen, method, or composition.

“Individual” or “animal” or “patient” or “mammal,” is meant any subject, particularly a mammalian subject, for whom any of the claimed compositions and methods is needed or may be beneficial. In preferred embodiments, the individual is a human. In more preferred embodiments, the individual is an adult human, i.e., at least 18 years of age.

“On-treatment” means any time point during treatment with an IFN-α, e.g, between the first and last doses, at which the skilled artisan would expect to observe an effect of the IFN-α on the level of a biomarker of the invention. Typical on-treatment time points include, e.g., one week, two weeks, four weeks, eight weeks, sixteen weeks, 30 days, 60 days, 90 days, 120 days, etc., after the first dose. The optimal on-treatment time point will typically vary depending on the disease, the identity of the biomarker, the bioactivity and dose of the IFN-α, and the expected time for IFN-α treatment to affect the level of the biomarker. For example, in evaluating a patient's sensitivity to IFN-α for treatment of resected, stage II or stage III melanoma based on reduced level of neutrophils, blood samples for testing for the development of grade 2 neutropenia would typically be drawn as early as about 3 weeks (or about 21 days) after the first dose, and if the result was negative for grade 2 neutropenia, additional blood samples would be taken once a week (or about every 7 days) thereafter until the patient either tested positive or had been on IFN-α therapy for about 16 weeks (or about 112 days).

“Parenteral administration” means an intravenous, subcutaneous, or intramuscular injection.

“Pharmaceutically acceptable” refers to molecular entities and compositions that are “generally regarded as safe” —e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

“Pre-treatment” means any time point before administration of the first dose of an IFN-α that would be useful to obtain a baseline measurement of a biomarker of the invention. Typical pre-treatment time points include, e.g., 24, 36, 48, or 72 hours, or one week, two weeks, etc., prior to the first dose. The optimal pre-treatment time point will typically vary depending on the disease, the identity of the biomarker, and the amount of time required to obtain the results of the baseline measurement.

“Treat” or “Treating” means to administer a therapeutic agent, such as a composition containing any of the interferon alfa proteins described herein, internally or externally to an individual in need of the therapeutic agent. Individuals in need of the agent include individuals who have been diagnosed as having, or at risk of developing, a condition or disorder susceptible to treatment with the agent, as well as individuals who have, or are at risk of developing, one or more adverse effects of treatment with a first therapeutic agent that are susceptible to alleviation with a second therapeutic agent. Typically, the therapeutic agent is administered in a therapeutically effective amount, which means an amount effective to produce one or more beneficial results. The therapeutically effective amount of a particular agent may vary according to factors such as the disease state, age, and weight of the patient being treated, and the sensitivity of the patient, e.g., ability to respond, to the therapeutic agent. Whether a beneficial or clinical result has been achieved can be assessed by any clinical measurement typically used by physicians or other skilled healthcare providers to assess the presence, severity or progression status of the targeted disease, symptom or adverse effect. Typically, a therapeutically effective amount of an agent will result in an improvement in the relevant clinical measurement(s) over the baseline status, or over the expected status if not treated, of at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%. While an embodiment of the present invention (e.g., a treatment method or article of manufacture) may not achieve the desired clinical benefit or result in every patient, it should do so in a statistically significant number of patients as determined by any statistical test known in the art such as the Student's t-test, the chi²-test, the U-test according to Mann and Whitney, the Kruskal-Wallis test (H-test), Jonckheere-Terpstra-test and the Wilcoxon-test.

### II. General.

The present invention provides pre-treatment and on-treatment biomarkers that are predictive of whether an individual is likely to have a beneficial response to IFN-α therapy. These IFN-α sensitive biomarkers are useful in selecting the patient population for whom an IFN-α composition is indicated, i.e., patients who are more sensitive to the beneficial effects of the IFN-α in the treatment of any disease that is susceptible for treatment with the IFN-α, and in monitoring the efficacy of IFN-α therapy during treatment.

### Pre-Treatment IFN-α Sensitivity Biomarkers

One class of biomarkers of the present invention relate to markers of the inflammatory status of an individual at baseline, i.e., prior to treatment with an IFN-α. In one embodiment, the biomarker is a human acute phase protein selected from the group consisting of: C-reactive protein (CRP), D-dimer, alpha 1-Antitypsin (A1AT, also referred to as serum trypsin inhibitor and alpha-1 proteinase inhibitor), alpha 1-antichymotrypsin, fibrinogen, thrombin (also referred to as activated Factor II [IIa], Factor VIII (FVIII), Von Willebrand factor (vWF), plasminogen (PLG), any one or more of the complement factors, ferritin. Serum amyloid P component (SAP), any one or more of the acute phase serum amyloid A proteins (A-SAAs), alpha-1-acid glycoprotein (AGP, also referred to as orosomucoid [ORM]), ferroxidase (also known as iron(II):oxygen oxidoreductase and ceruloplasmin and Haptoglobin (Hp).

In a preferred embodiment of the invention, the pre-treatment IFN-α sensitivity biomarker is an elevated level of high sensitivity C-reactive protein (CRP) in serum. CRP is an acute phase protein that appears in circulation in response to inflammatory cytokines, such as interleukin-6, and serves as a sensitive, though nonspecific, biomarker for systemic inflammation. Synthesized and released primarily by hepatocytes, CRP is a pentameric globular protein that has traditionally been used as a marker of infection and tissue injury. Serum CRP levels, which in apparently healthy individuals are typically less than 10 mg/L, may rise up to 3000-fold within 24-48 hours of an infectious or noninfectious stimuli. (Pepys, M B, Lancet 1:653-657 (1981)). However, low level increases in CRP have been reported in various conditions and disease states that may be associated with chronic, low-grade inflammation (see, e.g., Ridker, P. M. Circulation 107:363-369 (2003); Wasunna A. et al., Eur. J. Pediatr 149:424-427 (1990); Spector T. D. et al., Arthritis Rheum 40:723-727 (1997), Paul A. et al., Circulation 109:647-655 (2004), Allin, K. et al., Baseline C-Reactive Protein Is Associated With Incident Cancer and Survival in Patients With Cancer, J. Cin. Oncol., Mar. 16, 2009: doi:10.1200/JCO.2008.19.8440). Since CRP levels in apparently healthy individuals can be below 0.2 mg/L, high sensitivity assays for CRP (hsCRP assays) have been developed to detect low level changes in CRP levels. Thus, serum CRP levels measured by a hsCRP assay are referred to in the art and herein as serum hsCRP.

In a preferred embodiment, an individual is considered to test positive for an elevated hsCRP level if his/her hsCRP level is at least 1.0 mg/L; conversely a negative test for elevated hsCRP is an hsCRP level of <1.0 mg/L. Individuals having a hsCRP level that is >3.0 mg/L are likely to achieve a greater clinical benefit from IFN-α therapy than individuals having an hsCRP level of 1.0 mg/L ≦3.0 mg/L. IFN-α therapy would be expected to provide reduced clinical benefit to most patients with a hsCRP test result of <1.0 mg/L; for such patients, treatment with a different therapeutic agent, either in addition to, or instead of, IFN-α therapy may be appropriate.

Measurement of serum hsCRP level may be carried out using any of a variety of hsCRP assays known in the art, provided that the assay is capable of reliably measuring CRP concentrations in serum or plasma samples within the range of <1.0 mg/L to 10.0 mg/L, and preferably is capable of measuring CRP concentrations as low as 0.15 mg/L. Commercially available assays useful in practicing the present invention typically employ immunoturbidimetric and immunonephelometric based techniques, see, e.g., Roberts W. L. et al. Clin Chem 46:461-468 (2000). A preferred hsCRP assay is one that has been approved for marketing by the United States Food and Drug Administration pursuant to a 510(k) application.

In another preferred embodiment of the invention, if a patient tests positive for pre-treatment hsCRP, the validity of the prediction may be assessed by determining the individual's hsCRP level after initiation of IFN-α therapy. Preferably, the on-treatment hsCRP level is tested at 4 weeks. If the hsCRP level decreases from the pre-treatment level, the treating physician and patient would have a greater confidence that continuted IFN-α therapy will be beneficial.

### On-Treatment IFN-α Sensitivity Biomarkers

Another class of biomarkers of the present invention relate to the adverse effects of IFN-α and thus are measured after initiation of IFN-α therapy.

In one embodiment, an on-treatment IFN-α sensitivity biomarker is an on-treatment reduction in the levels of one or more of the following blood cell types: neutrophils, erythrocytes, and platelets, monocytes, eosinophils, and basophils. In preferred embodiments, the reduction in the blood cell type equates to a Grade 2 adverse event, as defined in the National Cancer Institute (NCI) Common Toxicity Grading Criteria, established Mar. 31, 2003 and published Aug. 9, 2006. The NCI toxicity criteria are included in the definitions of adverse events below.

Neutropenia is a condition in which there is a lower-than-normal number of neutrophils in the blood. The stated normal range for human blood counts varies between laboratories, but a neutrophil count of 2.5-7.5×10⁹/L is a standard normal range. People of African and Middle Eastern descent may have lower counts, which are still normal and is diagnosed by determining the ANC or the absolute granulocyte count (AGC) in a blood sample obtained from the patient.

Grade 1 neutropenia: <LLN-1500/mm³ ANC or <LLN-1.5×10⁹/L AGC.

Grade 2 neutropenia: <1500-1000/mm³ ANC or <1.5-1.0×10⁹/L AGC.

Grade 3 neutropenia: <1000-500/mm³ ANC or <1.0-0.5×10⁹/L AGC

Grade 4 neutropenia: <500/mm³ ANC or <0.5×10⁹/L AGC

For diseases or conditions in which lower doses of an IFN-α are used, such as in the treatment of chronic HCV infection, the presence or absence of an IFN-α sensitivity biomarker may require higher ANC or AGC cut-off values to discriminate effectively between subjects who experience a genuine treatment-emergent neutropenia—defining them as more sensitive to the biological effect of the IFN-α—and those subjects without such treatment-emergent neutropenia.

The invention also contemplates the diagnosis of neutropenia may be performed using alternative measures that estimate the neutrophil count, such as a white blood cell (WBC) count. For example, neutrophils account for approximately 70% of all white blood cells (leukocytes).

Anemia is a condition in which there is a lower than normal number of red blood cells (erythrocytes) or hemoglogin level in the blood. For men anemia is typically defined as a hemoglobin level of less than 13.5 gram/100 ml and for women as a hemoglobin level of less than 12.0 gram/100 ml.

Grade 1 anemia: hemoglobin level of <LLN-10.0 g/100 ml.

Grade 2 anemia: hemoglobin level of <10.0-8.0 g/100 ml.

Grade 3 anemia: hemoglobin level of <8.0-6.5 g/100 ml.

Grade 4 anemia: hemoglobin level of <6.5 g/100 ml.

The invention also contemplates the diagnosis of anemia may be made on the basis of alternative measures such as a reduced red blood cell (RBC) count.

Thrombocytopenia is a condition in which there is a lower than normal number of platelets (thrombocytes) in the blood. Normal platelet counts range from 150,000 and 450,000 per mm³. One common definition of thrombocytopenia is a platelet count of less than 100,000 per mm³.

Grade 1 thrombocytopenia: platelet count of <LLN-75,000/mm³.

Grade 2 thrombocytopenia: platelet count of <75,000-50,000/mm³.

Grade 3 thrombocytopenia: platelet count of <50,000-25,000/mm³.

Grade 4 thrombocytopenia: platelet count of <25,000/mm³.

Monocytopenia is an abnormally low level of monocytes in the peripheral blood, i.e., less than 200/mm³.

Eosinopenia is a decrease in the number of eosinophils in the blood, which normally make up about 1 to 3% of peripheral blood leukocytes. The upper limit of the normal range is 350 cells/mm³.

Basopenia is a deficiency of basophils and is typically defined as a basophil count of less than 0.01×10⁹/L. This condition is usually detected using flow cytometry.

In another embodiment, serum hsCRP levels are measured before and after initiation of treatment with an IFN-α to test for the presence of an on-treatment IFN-α sensitivity biomarker. An individual who experiences a reduction of hsCRP from the baseline level, preferably by at least about 25% by week four, or by at least about 50% by week 24, of IFN-α therapy would be likely to achieve a more robust, sustained and pronounced clinical benefit from continued treatment with the IFN-α than an individual whose hsCRP levels do not appreciably change after initiation of treatment.

### Testing for IFN-α Sensitivity Biomarkers

A physician can determine whether a patient has one or more of the biomarkers of the invention by ordering a laboratory test that measures the level of the desired acute phase protein(s) or blood cell type(s) in a blood sample obtained from the patient. The blood sample may be drawn from the patient by the physician or a member of the physician's staff, or by a technician at a diagnostic laboratory. In some embodiments, the physician may choose to order tests for the levels of two or more acute phase proteins or two or more blood cell types in determining whether a patient is a good candidate for initial or continued therapy with an IFN-α.

The physician may determine whether the level of the measured acute phase protein(s) or blood cell type(s) is abnormally high or low, respectively. This determination is based on the physician's sound judgment and can be based on comparison with reference values, such as from healthy individuals, from individuals with the same disease or condition, or criteria (e.g., toxicity criteria) established by a medical organization or regulatory agency. The reference values may also be set forth in the labeling, and/or in the prescribing information, for the IFN-α product to be used for the IFN-α based therapy. Alternately, the diagnostic laboratory may assign the patient as testing positive or negative for the biomarker based on comparing the measured level(s) of the acute phase protein(s) or blood cell type(s) to the appropriate reference values, and then provide a report to the patient and/or physician that states that the patient tested positive or negative for IFN-α sensitivity biomarker, with the report preferably including the numerical values for the levels of the acute phase protein(s) or blood cell type(s).

In deciding what acute phase proteins and blood cell types to test, or in deciding how to use the test results in treating any individual patient, the physician may also take into account other relevant circumstances, such as the disease or condition to be treated, the age, weight, gender, genetic background and race of the patient, and whether the patient is taking other therapeutic agents that could affect the levels of the acute phase protein(s) or blood cell type(s).

In some embodiments of the invention, the individual is tested prior to initiation of IFN-α therapy for a pre-treatment IFN-α sensitivity biomarker and again during IFN-α therapy for the presence of an on-treatment sensitivity biomarker.

### IFN-α Treatment

The IFN-α used in the compositions and methods of the present invention may be any of the multiple subtypes of IFN-α proteins expressed in humans and many other species (Pestka, S. et al., Immunol. Reviews 202:8-32 (2004); Diaz, M. O., et al., J. Interferon Cytokine Res 16:179-180 (1996). In preferred embodiments, the IFN-α protein is a recombinantly produced protein that consists of, or consists essentially of, the mature amino acid sequence for one of the following human IFN-α subtypes: IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21 (Bekisz, J. et al., Growth Factors 22(4):243-351 (2004)), as well as allelic variants for any of these subtypes, e.g., IFN-α2a, IFN-α2b, and IFN-α2c. Human IFN-α subtypes share 75-99% amino acid sequence identity and a mature sequence of 166 a.a. except for IFN-α2, which has 165 a.a. due to a deletion at position 44 (Bekisz, J., et al., supra). Other recombinant IFN-α proteins contemplated for use in the present invention include any consensus IFN-α protein in which the amino acid sequence has been designed by selecting at each position the amino acid that most commonly occurs at that position in the various native IFN-α subtypes.

Particularly preferred IFN-α compositions for use in the compositions and methods of the present invention are interferon alfa-2 products approved by a government regulatory agency, including any of the following: Roferon®-A (Interferon-alfa 2A, recombinant) marketed by Hoffmann La-Roche, Nutley N.J.), and pegylated versions thereof, such as PEGASYS® (peginterferon alfa-2a) marketed by Hoffmann La-Roche, Nutley N.J.); INTRON® A (Interferon alfa-2b, recombinant) marketed by Schering Corporation, Kenilworth, N.J.) and pegylated versions thereof, such as PegIntron® (peginterferon alfa-2b); (INFERGEN® (Interferon alfacon-1), a consensus IFN-α originally developed by Amgen, Thousand Oaks, Calif. and currently marketed by Three Rivers Pharmaceuticals, Warrendale, Pa. Other interferons contemplated for use in the present invention include fusions between interferon alfa and a non-interferon protein, such as Albuferon® (albinterferon alfa-2b) which is being developed by Human Genome Sciences, Rockville, Md. and Norvartis, Basel, Switzerland. These FN-α compositions may also be sold under different trade names, such as VIRAFERONPEG, which is the same composition as PegIntron® (peginterferon alfa-2b).

PEGASYS® (peginterferon alfa-2a) is obtained by covalent binding of one 40 kDa branched PEG-polymer via an amide bond to a lysine side chain of an interferon alfa-2b molecule, see, e.g., Dhalluin, C. et al., Bioconjugate Chem. 16:504-517 (2005) and U.S. Pat. No. 7,201,897. The resulting product is a mixture of mainly six monopegylated positional isomers (Dhalluin, C., supra, Foser, S. et al., J. Prot. Exp. Purif. 30: 78-87 [2003]). PEGASYS® (peginterferon alfa-2a) and biosimilars thereof are also referred to herein as bPEG40K-interferon alfa-2a.

PegIntron® (peginterferon alfa-2b) is obtained by covalently reacting recombinant interferon-alfa 2b with a succinimidylcarbonate PEG having an average molecular weight of 12,000 Da (SC-PEG12k) in 100 mM sodium phosphate, pH 6.5 (see, e.g., Grace, M. et al., J. Interferon Cytokine Res. 21:1103-1115 (2001); Wang, Y. S. et al., Adv. Drug Delivery Rev. 54:547-570 (2000); and U.S. Pat. No. 5,951,974). The resulting product is a mixture of mainly monopegylated species in which the PEG12k is attached to different residues of interferon alfa-2b via a urethane bond, with the majority positional isomer having the urethane bond at Histidine 34 (see, e.g., Wang, Y. S. et al., supra and U.S. Pat. No. 5,951,974). PegIntron® (peginterferon alfa-2b) and biosimilars thereof are also referred to herein as PEG12k-interferon alfa-2b.

Other IFN-α products contemplated for use in the invention that have been approved previously or are currently marketed, include: Berofor® alpha 2 (recombinant interferon alpha-2C, Boehringer Ingelheim Pharmaceutical, Inc., Ridgefield, Conn.; interferon alpha-n1, a purified blend of natural alfa interferons known as Surniferon® (Sumitomo, Japan) or as Wellferon® interferon alpha-nI (INS), Glaxo-Wellcome Ltd., London, Great Britain; a consensus alpha interferon such as those described in U.S. Pat. Nos. 4,897,471 and 4,695,623 (especially Examples 7, 8 or 9 thereof); ALFERON N Injection® [Interferon alfa-n3 (human leukocyte derived), a mixture of multiple species of natural alfa interferons available from Hemispherx Biopharma, Inc., Philadelphia, Pa.

Other interferon alfa-polymer conjugates useful in the present invention are described in U.S. Pat. No. 4,766,106, U.S. Pat. No. 4,917,888, European Patent Application No. 0 236 987, European Patent Application Nos. 0 510 356, 0 593 868 and 0 809 996 and International Publication No. WO 95/13090.

Pharmaceutical compositions of pegylated interferon alfas intended for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose, trehalose), carriers (e.g. human serum albumin), toxicity agents (e.g. NaCl), preservatives (e.g. thimerosol, cresol or benylalcohol), and surfactants(e.g. tween or polysorbates) in sterile water for injection. See, e.g., U.S. Pat. No. 6,180,096 and International Patent Application WO2006/020720. Such compositions may be stored as lyophilized powders under refrigeration at 2°-8° C. and reconstituted with sterile water prior to use. Such reconstituted aqueous solutions are typically stable when stored between and used within 24 hours of reconstitution. See, for example, U.S. Pat. Nos., 4,492,537; 5,762,923 and 5,766,582. Lyophilized pegylated interferon formulations may be provided in a pen-type syringe system that comprises a glass cartridge containing a diluent (i.e., sterile water) in one compartment and the lyophilized pegylated interferon-alfa powder in a separate compartment.

Examples of aqueous pegylated interferon formulations are described in U.S. Pat. No. 5,762,923. Such formulations may be stored in prefilled, multi-dose syringes such as those useful for delivery of drugs such as insulin. Typical suitable syringes include systems comprising a prefilled vial attached to a pen-type syringe such as the NOVOLET Novo Pen available from Novo Nordisk, as well as prefilled, pen-type syringes which allow easy self-injection by the user.

Diseases Susceptible to Treatment with IFN-α

Diseases and conditions that may be treated in accordance with the present invention are generally those that are susceptible to treatment with an IFN-α, i.e., the IFN-α achieves a clinically measurable benefical result. Exemplary diseases and conditions susceptible to treatment with an IFN-α include but are not limited to diseases caused by cell proliferation disorders, in particular cancers, and viral infections. Preferably, the disease is one for which the IFN-α has been approved by a regulatory agency such as the U.S. Food and Drug Administration.

Cancers include melanoma, chronic myelogenous leukemia (CML), renal cell cancer (RCC), hairy cell leukemia, Kaposi's sarcoma, multiple myeloma, basal cell carcinoma, malignant melanoma, superficial bladder cancer (SBC), ovarian cancer, follicular lymphoma, non-Hodgkin's lymphoma, cutaneous T cell lymphoma, condyloma accuminata, mycosis fungoides, carcinoid syndrome, colorectal cancer, laryngeal papillomatosis, and actinic keratosis. Preferred cancers and dosing regimens therefore are described in the regimens for chronic hepatitis C described in the labeling and prescribing information for the Roferon®-A (Interferon-alfa 2A, recombinant) and INTRON® A (Interferon alfa-2b, recombinant) products (see the Appendices attached hereto).

In preferred embodiments, the biomarkers of the present invention are used in conjunction with a pegylated IFN-α for treating patients with melanoma, chronic myelogenous leukemia (CML) or renal cell cancer (RCC), including, e.g., the treatment regimens described in U.S. Pat. No. 6,923,966 (melanoma), U.S. Pat. No. 6,605,273 (RCC) and U.S. Pat. No. 6,362,162 (CML); Bukowski R., et al., Cancer 95(2):389-396 (2002); Bukowski R., et al., J. Clin Oncol. 20(18):3841-348 (2002); Garcia-Manero, G. et al., Cancer 97(12):2010-2016 (2003); Garcia-Manero, G. et al., Cancer 98(3): 437-457 (2003); Michallet, M. et al., Leukemia 18:309-315 (2004); Motzer, R. J. et al., J. Clin Oncol. 19(5):1312-1319 (2001); Motzer, R. J. et al., Ann. Oncol. 13:1799-1805 (2002); Lipton, J. H., et al., Blood 100:782a Abstract 3091 (2002); Hochhaus, A., et al., Blood 100:164a Abstract 616 (2002); and Dummer et al., Proc. Am. Soc. Clin. Oncol. 22:712 Abstract 2861 (2003).

In one preferred embodiment, the biomarkers of the invention are used to identify patients with high-risk melanoma who are good candidates for IFN-α therapy, especially patients with Stage IIB (lesions>4 mm, but without positive nodes) and Stage III (lesions>4 mm and node-positive) primary cutaneous melanoma. Preferably the IFN-α therapy is used as adjuvant therapy after the patients have had surgery for their Stage IIB or Stage III melanoma. The biomarkers of the present invention will aid the treating physician in devising more efficacious treatment regimens for melanoma patients by helping the physician identify whether a patient is more likely to benefit from IFN-α therapy, either before or soon after beginning the therapy. Moreover, patients who test positive for a biomarker of the invention may be more willing to tolerate the side effects of IFN-α therapy.

In more preferred embodiments, the IFN-α used as adjuvant therapy is a pegylated IFN-α. The melanoma patients treatable in accordance with the improved methods of the present invention include those newly diagnosed with this disease who were free of disease post surgery but at high risk for systemic recurrence of the disease. The term “high risk patients” as used herein means those melanoma patients with lesions of Breslow thickness >4 mm as well as those patients with lesions of any Breslow thickness with primary or recurrent nodal involvement. Treatment with a pegylated IFN-α in accordance with the present invention will continue for up to five years, unless there is clinical evidence of disease progression, unacceptable toxicity or the patient requests that the therapy be discontinued.

When the pegylated IFN-α used for treating a high-risk melanoma patient is a PEG12k-interferon alfa-2b such as PegIntron® (peginterferon alfa-2b), the treatment regimen comprises administering to the patient a starting dose of about 3.0 to about 9.0 micrograms per kilogram once a week (QW), preferably in the range of about 4.5 to about 6.5 micrograms per kilogram QW, more preferably in the range of about 5.5 to about 6.5 micrograms per kilogram QW, and most preferably in the range of about 6.0 micrograms per kilogram QW. In some preferred embodiments, the high-risk melanoma patient is treated initially with 6.0 micrograms per kilogram of the PEG12k-interferon alfa-2b QW for eight weeks, and then with 3.0 micrograms per kilogram or less of the PEG12k-interferon alfa-2b QW for a period of five years minus the eight weeks of initial treatment. If less than 3.0 micrograms per kilogram are dosed to the patient, e.g., to maintain patient tolerance to the treatment, the dose is preferably reduced by 1 microgram per kilogram for each reduction, e.g., 3.0 to 2.0 to 1.0.

When the pegylated IFN-α used for treating a high-risk melanoma patient is a bPEG40K-interferon alfa-2a such as PEGASYS® (peginterferon alfa-2a), the treatment regimen comprises administering to the patient a dose of about 50 micrograms to about 500 micrograms QW, preferably about 200 micrograms to about 250 micrograms QW.

Viral infections include hepatitis A, hepatitis B, hepatitis C, hepatitis D, other non-A/non-B hepatitis, herpes virus, Epstein-Barr virus (EBV), cytomegalovirus (CMV), herpes simplex, human herpes virus type 6, papilloma, poxvirus, picornavirus, adenovirus, rhinovirus, human T lymphotropic virus-type 1 and 2, human rotavirus, rabies, retroviruses including human immunodeficiency virus (HIV), encephalitis and respiratory viral infections. In preferred embodiments, the viral infection is HCV or HBV. In a particularly preferred embodiment, the viral infection is HCV.

In preferred embodiments, the biomarkers of the present invention are used in conjunction with any IFN-α monotherapy or combination therapy treatment regimen approved by a regulatory authority for an HBV or HCV indication, and in particularly preferred embodiments, in conjunction with any of the dosing and treatment regimens for chronic hepatitis C described in the Package Inserts for the Roferon®-A (Interferon-alfa 2A, recombinant), PEGASYS® (peginterferon alfa-2a), INTRON® A (Interferon alfa-2b, recombinant) and PegIntron® (peginterferon alfa-2b) products (see the Appendices attached hereto). Approved combination therapy regimens for HCV use ribavirin in addition to the IFN-α protein. The biomarkers of the present invention may also be used to select patients who are likely to benefit the most from treatment with investigational combination regimens for HCV that add a small molecule inhibitor of the HCV protease and/or a small molecule inhibitor of the HCV polymerase to Peg-IFN-α/ribavirin therapy.

HCV protease inhibitors useful in such combination regimens are described in published international application nos. WO2009/038663, WO 2007/092616, and WO 2002/18369 and in published U.S. patent application no. 2007/0042968. Preferred HCV protease inhibitors for use in combination regimens are boceprevir (Schering-Plough), telaprevir (Vertex) and ITMN-191 (R7227) (Intermune and Roche). HCV polymerase inhibitors useful in such combination regimens are described in Preferred HCV polymerase inhibitors are the NS5B polymerase inhibitor ITMN-8020 (Intermune), R1626 (Roche), ABT-333 and ABT-072 (Abbot).

### EXAMPLES

The following examples are provided to more clearly describe the present invention and should not be construed to limit the scope of the invention.

### Example 1.

### Identification of On-Treatment Grade 2 Neutropenia as an IFN-α Sensitivity Biomarker for Treatment of Melanoma with a Pegylated Interferon Alfa.

To test the hypothesis that on-treatment reduction in a blood cell type might be a marker of the host baseline non-specific immune status, and thus predictive of a beneficial response to IFN-α therapy, the inventor herein analyzed certain data from EORTC 18991, which was a prospective, randomized 1:1 phase 3 study that enrolled 1256 subjects after surgery for high-risk cutaneous melanoma and allocated them to observation or weekly treatment with PegIntron® (peginterferon alfa-2b. The primary study endpoint was relapse-free survival (RFS), or the time from randomization to first relapse at any anatomical site or death, whichever occurred first. A secondary efficacy endpoint was overall survival (OS). Further details of the study are described in (Eggermont A. M. M. et al., Lancet 372:117-126 [2008]).

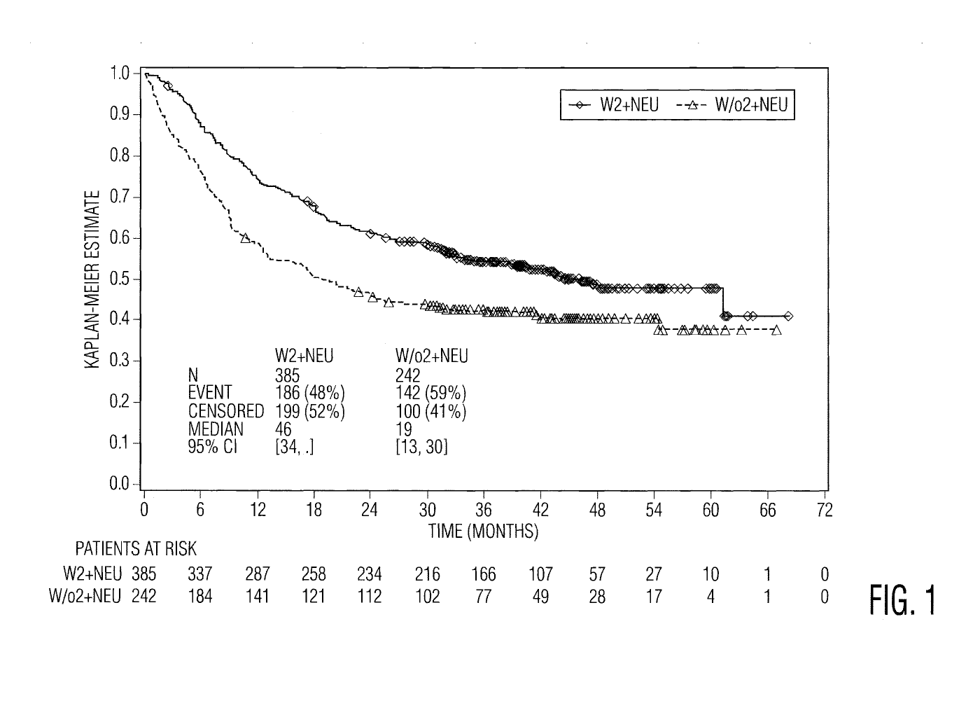

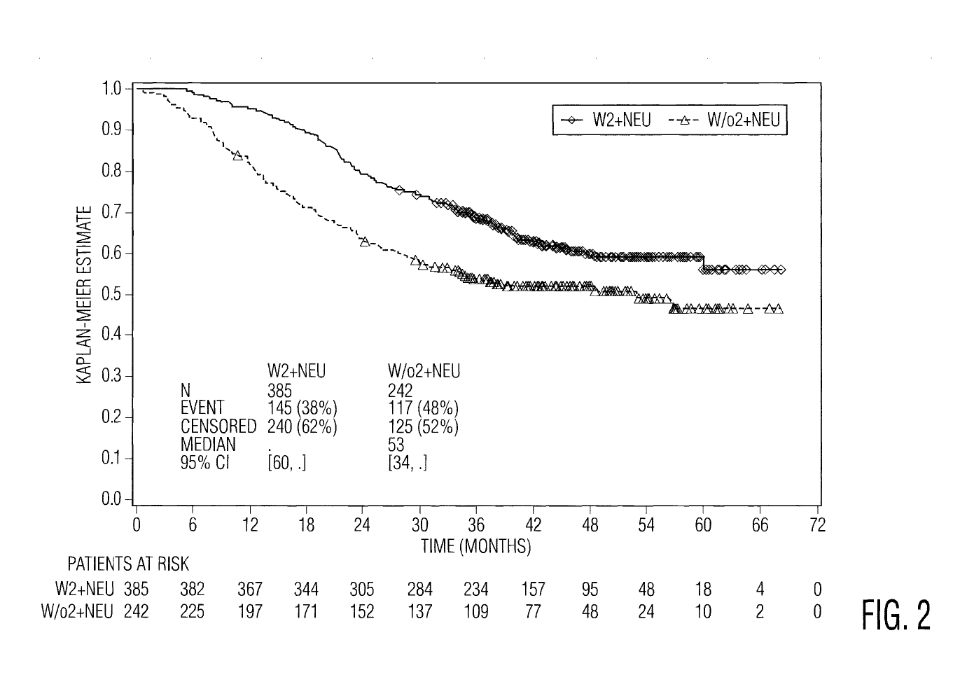

The inventor compared the RFS and OS outcomes in all 627 patients randomized for treatment with PegIntron® (peginterferon alfa-2b with the presence or absence of grade 2 or higher (grade 2+) neutropenia at any time point following initiation of treatment. In this analysis, patients with grade 1 neutropenia (barely abnormal value) were grouped together because grade 1 is about ½ case in deviation from normal and is also observed with no treatment intervention. Thus, grade 1 neutropenia would not discriminate effectively. Subjects with no lab values for neutrophil counts were considered as not having a grade 2+ neutropenia. The results are shown in FIGS. 1 and 2.

In FIG. 1, relapse-free survival (RFS), the primary efficacy and clinical benefit outcome, is represented as a Kaplan-Meier (KM) plot. RFS, based on independent review committee adjudication of the primary measure variable, was defined as the time from randomization to melanoma relapse at any anatomical site(s) or death from any cause, whichever occurred first. On the x axis is the time from randomization, in months. On the y axis is the actuarial probability of staying alive and free from relapse. The solid line represents those subjects in this group who experienced at least one episode of grade 2 or worse neutropenia (denoted as W2+ NEU), the dotted line is for subjects who did not have grade 2+ neutropenia or for whom neutrophil count data was lacking as discussed above (denoted as W/o2+ NEU). Triangles represent censoring in this KM analysis. At the bottom of the graph are provided the numbers of subjects at risk at various time points (in months).

This KM analysis identified 385 subjects who were in the W2+ NEU group and at risk of relapse and 242 subjects were in the group of W/o2+ NEU and at risk of relapse. The hazard ratio point estimate (95% confidence interval) is 0.67 (0.54-0.84) in favor of W2+ NEU, indicating an overall risk reduction of 33% for relapse or death if the subject was W2+ NEU compared to subjects in the W/o2+ NEU group.

FIG. 2 is a KM plot of the analysis comparing OS outcomes, which was defined as the time from randomization to death from any cause, and the presence or absence of grade 2 or higher neutropenia. On the x axis is the time from randomization, in months. On the y axis is the actuarial probability of staying alive. The solid line represents those subjects who experienced at least one episode of grade 2 or worse neutropenia (noted as W2+ NEU), the dotted line is for subjects who grade 2+ neutropenia or for whom neutrophil count data was lacking as discussed above (denoted as W/o2+ NEU). Triangles represent censoring in this KM analysis. At the bottom of the graph are provided the numbers of subjects at risk at various time points (in months).

This KM analysis identified 385 subjects who were in the W2+ NEU group and at risk of relapse and 242 subjects were in the group of W/o2+ NEU and at risk of relapse. The hazard ratio point estimate (95% confidence interval) is 0.64 (0.50-0.81) in favor of W2+ NEU, indicating an overall risk reduction of 36% for death if in W2+ NEU compared to subjects in the W/o2+ NEU group.

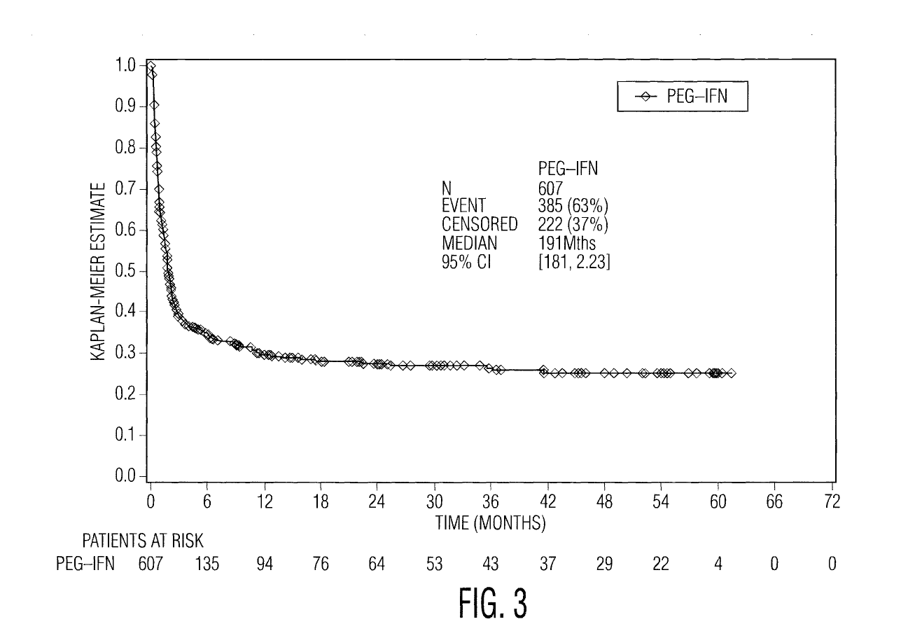

The inventor also analyzed the length of time between randomization, which was very close to the first dose of peginterferon alfa-2b, and the first observation of neutropenia of grade 2 or above in severity. This analysis was conducted for n=607 subjects randomized to and actually treated with peginterferon alfa-2b and included in the safety evaluable population. It does not account for the 20 subjects randomized to peg-IFN and who never received a single dose of peginterferon alfa-2b. The results of this analysis are shown in FIG. 3.

In the KM plot of FIG. 3, on the x axis is the KM estimate of time from randomization in months and in the y axis is the KM estimate of the actuarial proportion of not having observed the event first grade 2 or above neutropenia. In this analysis, the median time to first grade 2 or worse neutropenia is 1.91 months. In restricting the analysis to subjects who eventually had a grade 2 or worse neutropenia, the median time is 30 days, 75% subjects are discriminated by day 60 and by day 111 post randomization, 90% of those grade 2 or worse events were observed.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

1-30. (canceled)

31. A composition comprising an interferon alfa (IFN-α) for treating an individual having a disease susceptible to treatment with the IFN-α and a positive test for at least one IFN-α sensitivity biomarker,
wherein the IFN-α sensitivity biomarker is an elevated pre-treatment level of an acute phase protein, a reduced on-treatment level of high sensitivity CRP (hsCRP) or a reduced on-treatment level of at least one blood cell type selected from the group consisting of: neutrophils, erythrocytes, platelets, monocytes, eosinophils, and basophils.

32. The composition of claim 1, wherein the disease susceptible to treatment with the IFN-α is a cancer or a viral infection and the IFN-α sensitivity biomarker is a reduced on-treatment level of neutrophils.

33. The composition of claim 2, wherein the disease susceptible to treatment with the IFN-α is a cancer and the positive test for a reduced on-treatment level of neutrophils is Grade 2, Grade 3 or Grade 4 neutropenia.

34. The composition of claim 1, wherein the IFN-α sensitivity biomarker is an elevated pre-treatment level of an acute phase protein, wherein the acute phase protein is C reactive protein (CRP) and the positive test for an elevated pre-treatment level of the acute phase protein is a serum high sensitivity CRP (hsCRP) concentration of 1 mg/liter or greater or a serum high sensitivity CRP (hsCRP) concentration of greater than 3 mg/liter.

35. The composition of claim 1, wherein the disease susceptible to treatment with the IFN-α is a cancer, the IFN-α sensitivity biomarker is a reduced on-treatment level of erythrocytes or platelets, the positive test for a reduced on-treatment level of erythrocytes is Grade 2, Grade 3 or Grade 4 anemia and the positive test for a reduced on-treatment level of platelets is Grade 2, Grade 3 or Grade 4 thrombocytopenia.

36. The composition of claim 1, wherein the disease susceptible to treatment with the IFN-α is melanoma, renal cell carcinoma (RCC), chronic myelocytic leukemia (CML), infection with hepatitis C virus (HCV), or infection with hepatitis B virus (HBV).

37. The composition of claim 6, wherein the disease susceptible to treatment with the IFN-α is infection with hepatitis C virus (HCV).

38. The composition of claim 6, wherein the disease susceptible to treatment with the IFN-α is melanoma.

39. The composition of claim 8, wherein the disease susceptible to treatment with the IFN-α is resected, high risk stage II or stage III cutaneous melanoma.

40. The composition of any of claims 1-9, wherein the IFN-α is an interferon alfa-2a protein, an interferon alfa-2b protein, an interferon alfa-2c protein or a consensus interferon alfa protein.

41. The composition of claim 10, wherein the interferon alfa-2a protein is a pegylated interferon alfa-2a protein or an albumin-interferon alfa-2a fusion protein and wherein the interferon alfa-2b protein is a pegylated interferon alfa-2b or an albumin-interferon alfa-2b fusion protein.

42. The composition of claim 11, wherein the disease susceptible to treatment with the IFN-α is resected, high risk stage II or III melanoma, the interferon alfa-2b protein is PegIntron® (peginterferon alfa-2b ), and the interferon alfa-2b protein is formulated for parenteral administration of a dose of 6.0 micrograms/kg or 3 micrograms/kg.

43. A method of predicting an individual's response to therapy with an interferon alfa (IFN-α) for treating a cancer or a viral infection, comprising obtaining a blood sample from the individual, assaying the blood sample for the presence of at least one interferon sensitivity biomarker, and making a prediction based on the results of the assaying step,
wherein if the results are positive for the presence of the assayed biomarker, the prediction is that the individual is likely to achieve a beneficial response, and if the results are negative for the presence of the assayed biomarker, the prediction is that the individual is not likely to achieve a beneficial response,
wherein the interferon sensitivity marker is an elevated pre-treatment level of an of an acute phase protein, a reduced on-treatment level of high sensitivity CRP (hsCRP) or a reduced on-treatment level of at least one blood cell type selected from the group consisting of: neutrophils, erythrocytes, platelets, monocytes, eosinophils, and basophils.

44. The method of claim 13, wherein the response to IFN-α therapy is for treating melanoma and the IFN-α sensitivity biomarker is a reduced on-treatment level of neutrophils and the positive test for a reduced on-treatment level of neutrophils is Grade 2, Grade 3 or Grade 4 neutropenia.

45. The method of claim 14, wherein the IFN-α is a pegylated interferon alfa-2a protein, an albumin-interferon alfa-2a fusion protein, a pegylated interferon alfa-2b or an albumin-interferon alfa-2b fusion protein.

46. A method of selecting a therapy for treating an individual having a disease susceptible to treatment with an interferon alfa (IFN-α), comprising testing the individual for the presence of at least one IFN-α sensitivity biomarker and selecting a therapy based on the results of the testing step, wherein if the individual tests positive for the IFN-α sensitivity biomarker, the selected therapy comprises initial treatment or continued treatment with the IFN-α and if the individual tests negative for the interferon sensitivity biomarker, the selected therapy comprises the IFN-α in combination with at least one other therapeutic agent that is not an IFN-α or the selected therapy excludes any IFN-α-based therapy,
wherein the interferon sensitivity biomarker is an elevated pre-treatment level of an of an acute phase protein, a reduced on-treatment level of high sensitivity CRP (hsCRP) or a reduced on-treatment level of at least one blood cell type selected from the group consisting of: neutrophils, erythrocytes, platelets, monocytes, eosinophils, and basophils.

47. The method of claim 16, wherein the disease susceptible to treatment with the IFN-α is a cancer or a viral infection and the IFN-α sensitivity biomarker is a reduced on-treatment level of neutrophils.

48. The method of claim 17, wherein the disease susceptible to treatment with the IFN-α is melanoma, the IFN-α is a pegylated interferon alfa-2a protein, an albumin-interferon alfa-2a fusion protein, a pegylated interferon alfa-2b or an albumin-interferon alfa-2b fusion protein and the positive test for a reduced on-treatment level of neutrophils is Grade 2, Grade 3 or Grade 4 neutropenia.

49. The method of claim 18, wherein the IFN-α is a pegylated interferon alfa-2b protein.

50. The method of claim 16, wherein the disease susceptible to treatment with the IFN-α is melanoma and the IFN-α sensitivity biomarker is an elevated pre-treatment level of an acute phase protein, wherein the acute phase protein is C reactive protein (CRP) and the positive test for an elevated pre-treatment level of the acute phase protein is a serum high sensitivity CRP (hsCRP) concentration of 1 mg/liter or greater or a serum high sensitivity CRP (hsCRP) concentration of greater than 3 mg/liter.
